(19) United States
(12) Patent Application Publication
(10) Pub. No.: US 2007/0197958 A1
(43) Pub. Date: Aug. 23, 2007

(54) Probe for insertion in the anal canal and rectum of a body

(75) Inventor: Soren Hern, Minsk (BY)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Contura SA, Lausanne (CH)

(21) Appl. No.: 11/525,216

(22) Filed: Sep. 22, 2006

Related U.S. Application Data
(60) Provisional application No. 60/719,192, filed on Sep. 22, 2005.
(60) Provisional application No. 60/725,669, filed on Oct. 13, 2005.

(30) Foreign Application Priority Data
Sep. 22, 2005 (DK) PA 2005 01324
Oct. 13, 2005 (DK) PA 2005 01432

(51) Int. Cl. A61M 1/06; A61M 31/00

(52) U.S. Cl. 604/73; 604/514

(57) ABSTRACT

The present invention relates to a probe for insertion in the anal canal (canalis analis) and rectum of a body for the purpose of examining and treating patients suffering from diseases therein. More specifically, the invention relates to a probe for use in treatment of patients suffering from anal incontinence by injection of a bulking agent for obtaining a restricted fecal/flatus passageway in the anal canal.

### CROSS-REFERENCE TO RELATED APPLICATIONS

This application claims the benefit of U.S. Provisional Application Nos. 60/719,192, filed Sep. 22, 2005, and 60/725,669, filed Oct. 13, 2005. This application also claims priority to Denmark Application Nos. PA 2005 01324, filed Sep. 22, 2005, and PA 2005 01432, filed Oct. 13, 2005. The aforementioned applications are hereby incorporated by reference in their entirety.

### BACKGROUND OF THE INVENTION

Anal incontinence disease is a widespread medical problem for many people, and several kinds of medical and surgical treatment are developed and used for curing these people. For example different ways of anal sphincter surgical repair are used to improve the function of the sphincter, artificial anal sphincter devices can be implanted in the patient preventing fecal/flatus leakage or a bulking agent can be injected into the tissue of the anal canal for restricting the fecal passageway. The present invention is preferably used in connection with the latter method of injecting a bulking agent.

The outer layer of the anal canal is covered mostly with mucosal tissue preventing a sufficient sterilisation of the area. Hence there is an increased risk of infections when inserting any material through the anal mucosa. Therefore, surgeons today preferably inject a bulking agent from the outside (through the muscle tissue of the buttock) and not via the anal canal for minimising the risk of injuring and infecting it. However, it is extremely difficult to determine the exact position to place a quantity of a bulking agent even using ultrasound control using the prior art technique.

Furthermore, it is important for surgeons in medical practise, whether it is for the purpose of examining or surgical procedures, to easily inspect and/or interact with the patient's organs and cavities in order to obtain the most successful and comfortable procedure for the patients, which is not the case with present methods.

An object of the present invention is to provide a device for use in treatment of the anal canal via anus.

It is a further object of the present invention to provide a device for use in treatment of the anal canal via anus, which minimises or even eliminates the risk of injuring the anal canal and in particular the rectum with the surgical instruments during examination and treatment.

It is a further object of the present invention to provide a device for use in treatment of the anal canal via anus, which eases the way of reaching the correct position to be treated.

It is a further object of the present invention to provide a less painful and faster method of examining and treating patient suffering from disorders in the anal canal.

### SUMMARY OF THE INVENTION

The above mentioned objects are achieved by a probe according to a first aspect of the invention, said probe for insertion in a patients anal canal and rectum comprising a hollowed tubular member having a closed distal end adapted to enter the anal canal and an oppositely arranged open end for receiving surgical instruments, at least one longitudinally extending groove being provided in the sidewall of the hollowed member so as to allow a part of the patient's anal canal to enter into the groove for examination and treatment, said groove being accessible for examination and treatment of said part of the anal canal via an opening near said open end of the hollowed member.

The mucosal tissue of the anal canal will fall into the groove in the tubular member. Thereby the tissues of the anal canal is placed in an excellent position in order to inject a bulking agent into the tissues between the mucosal tissue and the muscular tissue surrounding the anal canal, using e.g. a hollow cannula inserted into the tissue in the groove through said opening near said open end of the hollowed member. This constitutes a considerable advantage over the prior art with respect to position the bulking agent correctly.

The probe preferably comprises a head part defined by or at said closed distal end and being rotatable around an axis of rotation being parallel to the longitudinal centre axis of the probe, but not coincident therewith. Thus, the head of the probe can be rotated such that it can be positioned displaced in relation to the rest of the probe. As described below, this is particularly useful when measuring the length of the anal canal, which is important for the surgeon to know, so that he/she does not inject a bulking agent in the rectum but only in the anal canal.

The head part may be rotatable via a handle provided near the open end of the probe.

The probe is preferably provided with one or a set of measuring line(s) on its outer surface for use when measuring the length of the anal canal.

In one method according to the invention, a surgeon inserts the probe into the anal canal such and orients the probe such that the groove is situated adjacent to a site to be inspected and/or treated. The longitudinal orientation placement of the distal closed end of the probe is at linea dentalis in the anal canal or about 1 cm from the linea dentalis.

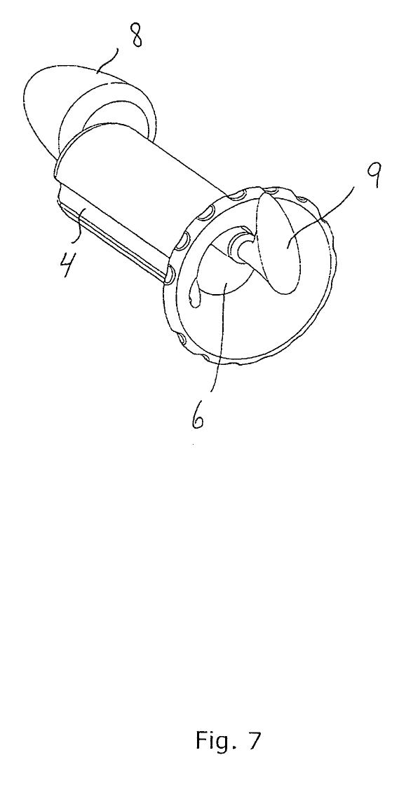

In another method according to the invention, the surgeon inserts the probe into the anal canal such that the head part of the probe enters into the rectum. Then the surgeon rotates the head part so that it is positioned displaced in relation to the other (main body) part of the probe (see FIG. 7). Then the surgeon retracts the probe until the head part abuts the distal end of the anal canal (at the junction to the rectum) and the measuring line is read so that the surgeon knows the exact length of the anal canal.

When the probe is inserted, a part of the tissue, i.e. mucosal tissue, of the anal canal will enter (sink) into the groove(s) of the probe (due to the material properties of this tissue), where it can be inspected and or treated. The hollowed part of the probe then provides space for the surgeon to introduce instruments for examining the anal canal while eliminating the risk of injuring and infecting the other parts of the anal canal or rectum, as the probe protects these. The part of the anal canal that is in the groove can then be treated by introducing instruments into the groove, which is accessible from the open end of the hollowed member and via said opening.

The probe may comprise a removable element (an obturator) positioned in said groove for filling out the groove, so that the probe has a smooth continuous outer surface during insertion and movement in the anal canal. Thus, the probe can be oriented correctly in the anal canal by rotating it until the correct tissue area to be treated is facing the groove. The movable element is preferably a pin-like member having the same cross-sectional shape as the groove.

The probe may be widened at the open end to ensure that the probe does not slide up into the anal canal and rectum, and the probe may comprise a collar arranged near said open end for defining an insertion stop for the probe.

As mentioned, the probe can be rotated in the anal canal for reaching the correct tissue area to be treated, which is useful, if different parts are to be treated. However, the probe may comprise more than one, such as two or three or four or more grooves, each extending longitudinally in said sidewall so as to allow parts of the patient's anal canal to enter into the groove for examination and treatment, when the probe is inserted therein. It is thereby possible to examine and treat various tissue areas without moving the probe inside the anal canal, once it has been placed.

Preferably, the groove(s) extends substantially from the closed end or head part to the open end, but its length may vary. In embodiments of the probe according to the invention having a rotatable head, the groove(s) does preferably not extend in the rotatable head part.

In an embodiment, the groove(s) does not extend through the sidewall of the hollowed member, i.e. there is no communication from the groove with the internal space of the hollow member, because this would not be convenient, in particular if the patient has a prolapsus of the anal canal, which then will enter into the hollowed space of the probe, and it will be difficult to retract if the tissue has been injected with a bulking agent.

However, in an alternative embodiment, the groove may go through the sidewall, so that a part of the anal canal enters into the hollowed space of the probe, as it is possible to use such a probe for certain patients.

The distal end of the probe defined by the closed end/the head part preferably has a substantially cone-shaped form for easing insertion. In any case the distal closed end is preferably rounded and smooth, including the transition from the end part to the main body part of the hollow member.

The elasticity of the anal canal varies from patient to patient, and normally the anal canal tissue will be less elastic and loose with increasing age of the patient. Therefore, the probe's dimensions depends on the patient to be treated. The probe (the main body part of the probe) is substantially cylindrical with an outer diameter of between 20 mm and 30 mm, and the width of the groove(s) is between 1 mm and 5 mm, preferably 3 mm. The length of the probe is preferably between 50 mm and 100 mm.

The open end and groove is adapted to receive surgical instruments for exercising and treatment of the anal canal, such as scopes, ultrasound probes or needles/cannulas.

According to another aspect, the present invention relates to the use of the probe for examination and surgical treatment of the anal canal of a mammalian body.

According to a further aspect, the present invention relates to a method of treating anal incontinence comprising the use of the probe. The method comprises the steps of; inserting the probe into the anal canal of a patient and injecting an agent into the part of the anal canal, which has entered the groove(s) of the probe.

The method may further comprise, prior to the step of injecting, turning the probe until the part of anal canal to be examined or treated is aligned with one or more of the grooves of the probe, retracting the removable element(s) filling out the groove(s) so as to allow that part of anal canal to enter the groove(s).

The method may further comprise, after the step of inserting, scanning the anal canal with an ultrasound probe inserted into the hollowed part of the probe. The probe is adapted to receive an ultrasound probe in the hollowed space, and the ultrasound preferably fills out the entire hollowed space. Thus, by use of the probe according to the invention, it is easy for the surgeon to examine the anal canal by use of an ultrasound probe.

The method may further comprise, prior to the step of injecting; rotating the head part into a displaced position in relation to the other part of the probe, retracting the probe out of the anal canal until said part abuts the tissue of the anal canal forming the junction to the rectum, and measuring the length of the anal canal by means of measuring marks provided on the probe. When the length of the anal canal has been measured, the head part may be rotated into an aligned position in relation to the other part of the probe, meaning the outer surface of the hollowed part of the probe, and the probe can then be retracted from the anal canal.

For the treatment of anal incontinence, the bulking agent can be a hydrogel, which is injected into the tissue surrounding the anal canal, specifically under the submucosal membrane. Injections of 2 to 6 ml are suitable. The hydrogel is preferably distributed at more than one cross-sectional position along a single longitudinal position of the anal canal. In a particular suitable embodiment, three or more depots are made along a single longitudinal position of the anal canal, preferably at positions 10, 2 and 6 o'clock on the cross-sectional axis of the anal canal.

The bulking agent is preferably injected via a hollow cannula inserted through the opening in the open end of the probe and through the tissue which has sunk into the groove.

The groove provides a guiding chute for guiding the instruments along the tissue of the anal canal, so as to ease the handling of the instruments inside the probe and for providing an even better preciseness for the injection of an agent in the tissue.

The probe is preferably disposable, and it may be market in a kit comprising the probe, a material suitable for injection, such as a bulking agent, and surgical instruments, such as a syringe and needle/cannula for injecting the material.

The probe is preferably made of plastic, and the probe or at least the groove of the probe is made of a transparent plastic that enables light to pass through, which results in a minimum of shadows in the probe and thus a better view of the anal canal.

According to a further aspect, the present invention relates to a probe for insertion in a patients anal canal and comprising a hollowed tubular member with an open end for receiving surgical instruments, a head part arranged opposite to said open end and being adapted to enter the anal canal, at least one longitudinally extending groove being provided in the sidewall of the hollowed member so as to allow a part of the patients anal canal to enter into the groove, said groove being accessible for examination and treatment of said part of the anal canal via an opening near said open end of the hollowed member.

Said head part may be defined by a closed end of said hollowed member. The head part may be rotatable around an axis of rotation that is substantially parallel to, but not coincident with the longitudinal centre axis of the probe.

The probe according to this last aspect may comprise any of the elements mentioned in connection with the first aspect of the invention.

It should be understood that the words “body” and “patient” cover any mammalian body, such as a human.

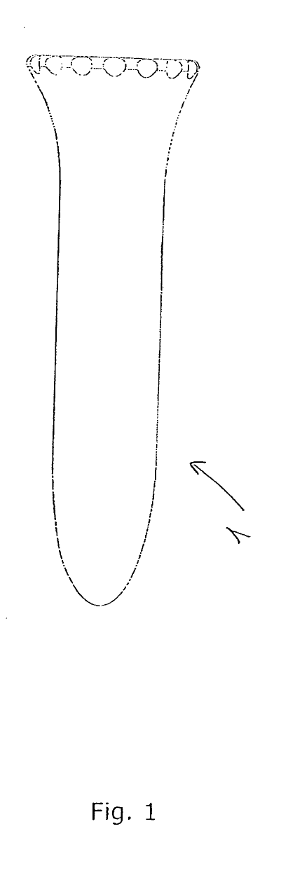

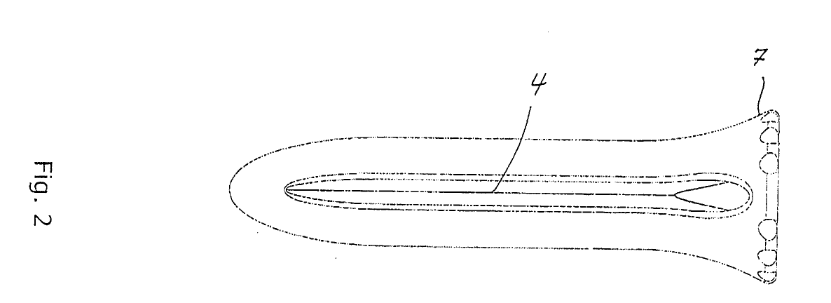

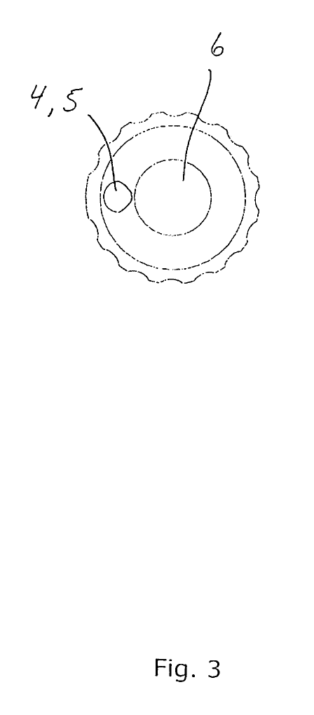

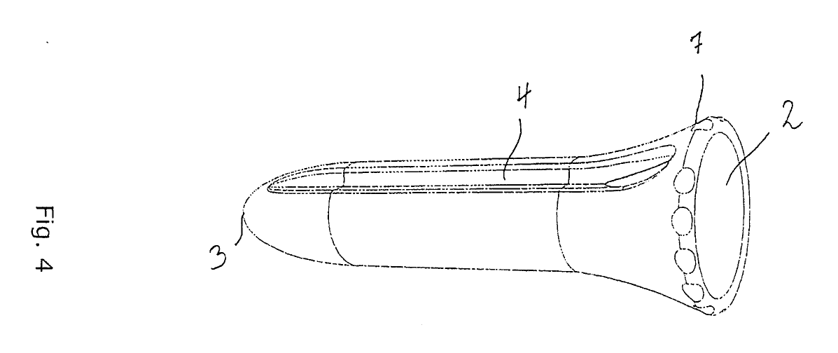

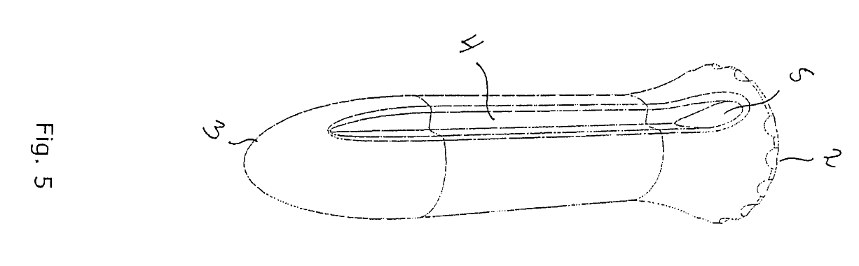

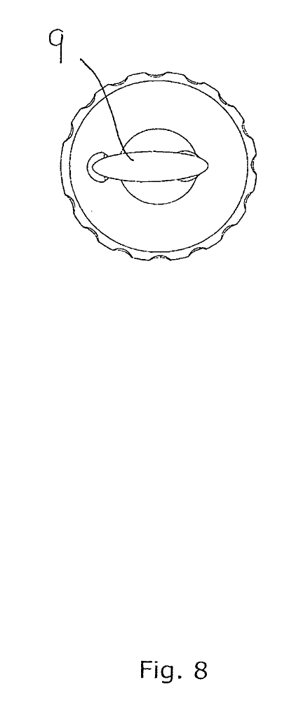

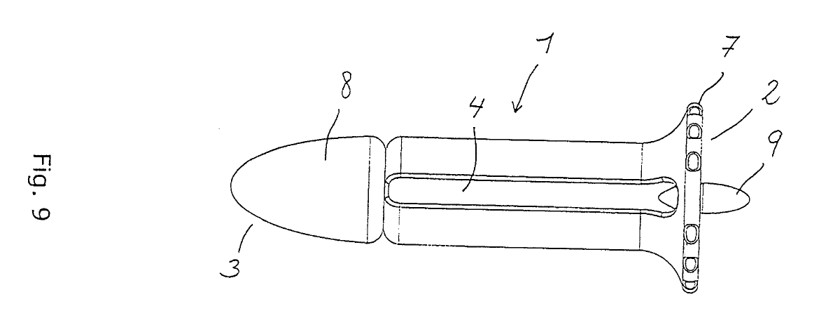

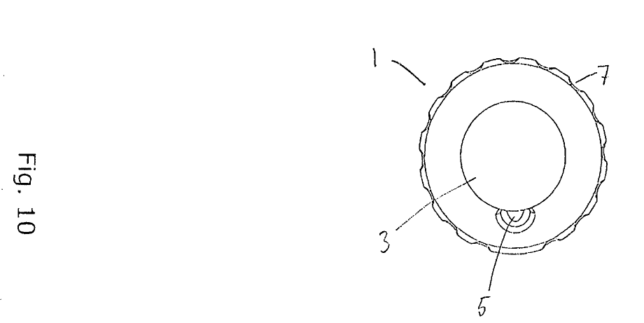

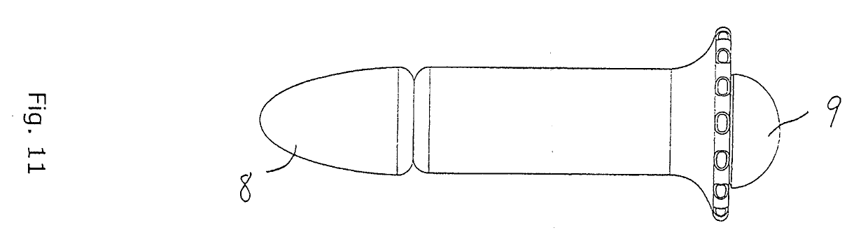

A preferred embodiment of the invention will now be described in detail with reference to the accompanying FIGS. 1-13, wherein

FIGS. 1-5 show a first embodiment of a probe according to the invention, and

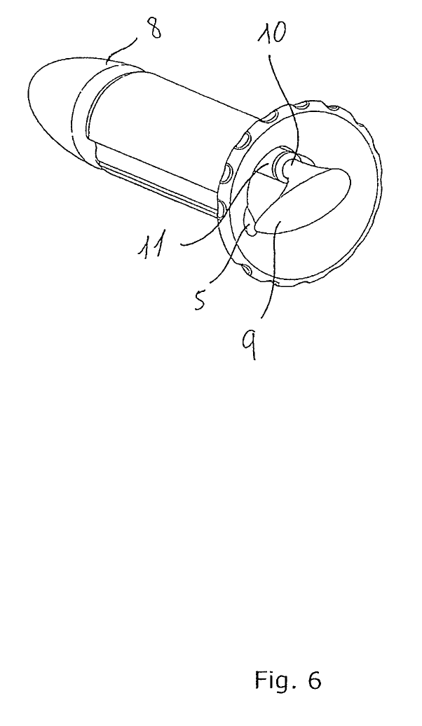

FIGS. 6-13 show a second embodiment of a probe according to the invention,

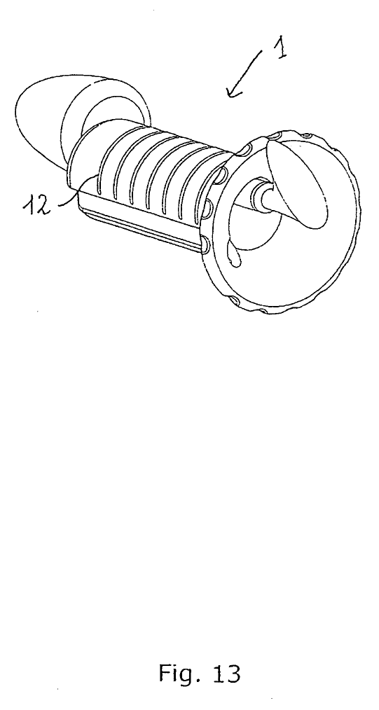

FIGS. 13-19 show a third embodiment of a probe according to the invention.

FIG. 1-5 shows an embodiment of the probe 1 according to the invention. The probe 1 has an open end 2 and a closed end 3, and an elongate main body part extending there between. The main body part of the probe has a circular cross-sectional shape, i.e. it is cylindrical, and the closed end is rounded, smooth and substantially cone-shaped for easing the insertion of the probe into the anal canal. The probe is hollow from the open end to the closed distal end. The elongate main body part thereby constitutes a sidewall of the probe.

The probe comprises a groove 4 provided in the sidewall. The groove 4 extends longitudinal in the sidewall along a length of the probe. When the probe is inserted in the anal canal, a part of the anal canal will enter (or lay/sink down) into the groove 4 so that a surgeon can examine and treat that part via the opening 5 and via the hollowed space 6 of the probe. Such treatment may comprise the injection of a bulking agent through a cannula inserted via an opening 5 in the widened (proximal) open end of the probe, which opening 5 communicates with said groove. The probe is preferably made of transparent plastic, so that it is easier to view anal canal in the hollowed space of the probe.

The probe 1 comprises a widened portion or collar 7 at the open end defining an “insertion stop” for the probe.

FIGS. 6-13 show a second embodiment of the probe 1, wherein the probe 1 comprises a rotatable head part 8. The head part 8 is rotated around an axis of rotation being parallel to, but not coincident with the longitudinal axis of the probe.

The head part 8 is rotated by means of a handle 9 provided at the open end 2 of the probe 1. The handle is connected to the head via a shaft 10 that is suspended in a tube 11 provided in the hollowed part of the probe. FIGS. 6 and 8-11 show the probe 1 in a condition where the head part 8 is aligned with the rest of the probe 1, and FIG. 7 shows the probe 1 in a condition where the head 8 is rotated into a displaced position in relation to the rest of the probe 1.

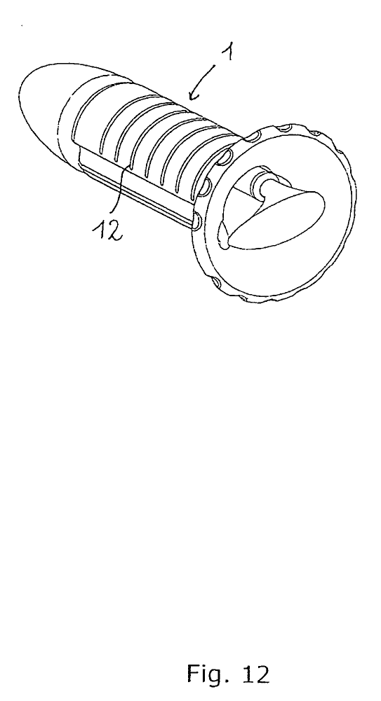

FIGS. 12 and 13 show the probe of FIGS. 6-11 comprising measuring marks/measuring lines 12 on the outer surface for measuring the length of the anal canal.

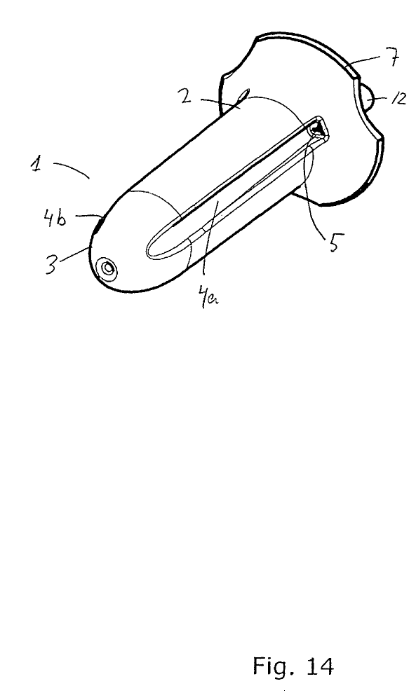

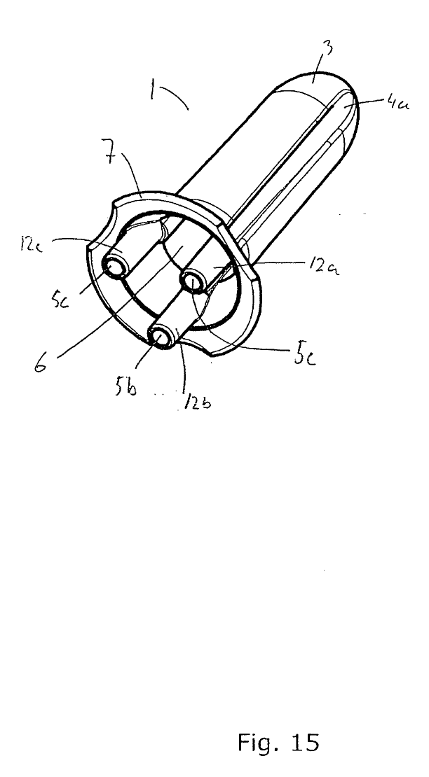

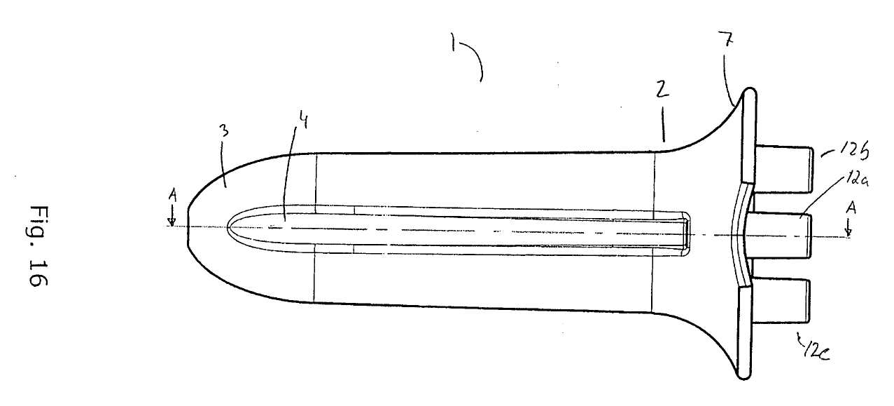

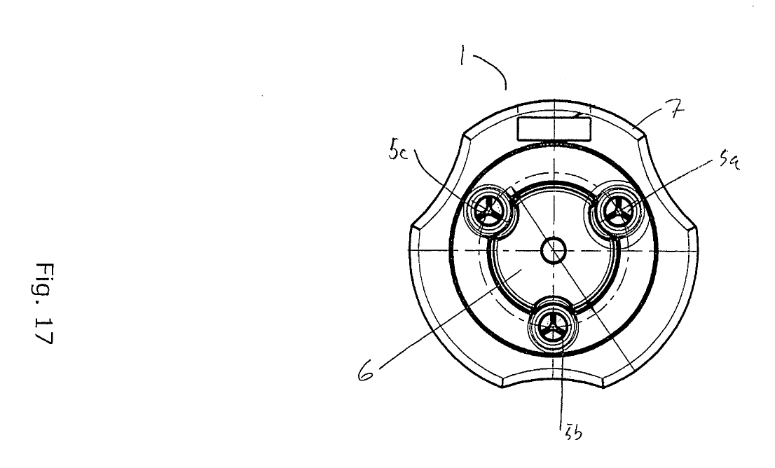

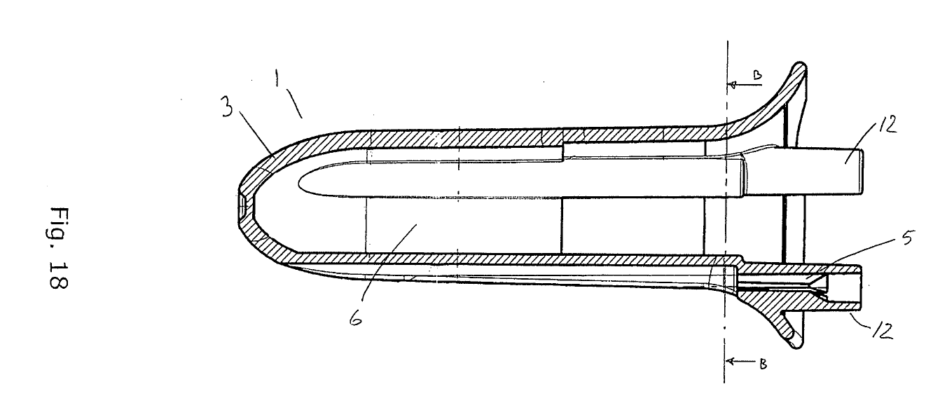

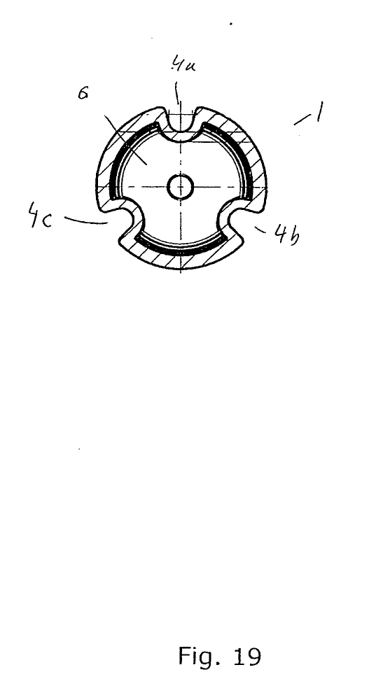

FIGS. 14 and 15 show a probe according to another embodiment of the invention in a perspective view from a distal and a proximal closed end respectively. The probe according to this embodiment is also shown in a side view in FIG. 16, and an end view (from the open proximal end 2) in FIG. 17. FIG. 18 shows a side sectional view (taken along A-A in FIG. 16) of the probe. FIG. 19 shows a cross section (taken along B-B in FIG. 18) through the main body part of the probe 1.

In many aspect the probe in this embodiment is similar to the probe described with respect to FIGS. 1-7, including shape and choice of material. Like reference numbers are used for like parts/features throughout the set of drawings.

The probe 1 in this embodiment (FIGS. 14-19) has three grooves 4a, 4,b, 4c, and altered stop 7, as compared with the embodiments shown in FIG. 1-13. The grooves extend in a direction parallel to the longitudinal axis of the probe, as was the case with the previously described embodiments. Whereas, the wall thickness of the main body part of the probe 1 shown in FIGS. 1-13 was thick enough to contain the grove(s) 4, in this embodiment the wall thickness has been reduced in order to improve visibility, such that it is easier for a surgeon to verify if a part of the tissue of the anal canal tissue wall has fallen/sunk into the groove(s) 4a, 4b, 4c. Thus, the grooves bulge into the space defined within the sidewalls of the main body part of the probe, as can be appreciated from FIG. 19.

In the embodiment shown the opening(s) 5a, 5b, 5c in the open end stop 7 of the probe has further been provided with a guide 12a, 12b, 12c for each opening 5a, 5b, 5c. The guide 12a, 12b, 12c serves the purpose of guiding a surgical tool, such as a syringe and needle/cannula, The guide(s) 12a, 12b, 12c are preferably provided with a luer connection, or other suitable connection means, adapted for receiving a distal end of a syringe or other injection device and dimensioned such that a cannula of this syringe/device can enter the grove 4a, 4b, 4c, through the opening 5.

The reduced wall thickness may also apply to probes having one, two, four or another number of grooves.

The reduced wall thickness may also apply to probes having other shapes of the stop 7, e.g. the one shown in the FIG. 1-13 embodiments.

A guide 12a, 12b, 12c may also be provide with the opening(s) 5 in the embodiments described above in relation to FIGS. 1-13, or other embodiments.

The invention concerns the following aspects:
a) A probe for insertion in a patients anal canal and comprising a hollowed tubular member having a closed end adapted to enter the anal canal and an oppositely arranged open end for receiving surgical instruments, at least one longitudinally extending groove being provided in the sidewall of the hollowed member so as to allow a part of the patients anal canal to enter into the groove, said groove being accessible for examination and treatment of said part of the anal canal via an opening near said open end of the hollowed member.
b) A probe according to aspect a) and comprising a rotatable head part defined by or at said closed end and being rotatable around an axis of rotation that is substantially parallel to, but not coincident with the longitudinal centre axis of the probe.
c) A probe according to aspect b) and comprising a handle near said open end for rotating said head part.
d) A probe according to aspect c), wherein the handle is connected to the rotatable head part via a shaft suspended inside the hollowed tubular member.
e) A probe according to any of aspects a)-d) and comprising a removable element positioned in said groove for filling out the groove during insertion and movement of the probe in the anal canal.
f) A probe according to any of aspects a)-e) and comprising a widened portion or a collar arranged near said open end for defining an insertion stop for the probe.
g) A probe according to any of aspects a)-f) and comprising two or three or four or more grooves distributed along the circumference of the hollowed member, each extending longitudinally in said sidewall so as to allow parts of the patients anal canal to enter into the groove for examining and treatment.
h) A probe according to any of aspects a)-g), wherein the closed end or head part has a substantially cone-shaped form for easing insertion.
i) A probe according to any of aspects a)-h), wherein the probe is substantially cylindrical with an outer diameter of between 20 mm and 30 mm.
j) A probe according to any of aspects a)-i), wherein the groove(s) extends substantially from the closed end or head part to the open end.
k) A probe according to any of aspects a)-j), wherein the width of the groove(s) is between 1 mm and 5 mm, preferably 3 mm.
l) A probe according to any of aspects a)-k), wherein the surgical instruments comprise scopes, ultrasound probes and/or needles.
m) A probe according to any of aspects a)-l), said probe being made of plastic.
n) A probe according to aspect m), wherein the probe or at least the groove(s) is made of transparent plastic.
o) A probe according to any of aspects a)-n) comprising a measuring line on its outer surface for measuring the length of the anal canal.
p) A probe for insertion in a patients anal canal and comprising a hollowed tubular member with an open end for receiving surgical instruments, a head part arranged opposite to said open end and being adapted to enter the anal canal, at least one longitudinally extending groove being provided in the sidewall of the hollowed member so as to allow a part of the patients anal canal to enter into the groove, said groove being accessible for examination and treatment of said part of the anal canal via an opening near said open end of the hollowed member.
q) A probe according to aspect p, wherein said head part is defined by a closed end of said hollowed member.
r) A probe according to aspect p) or q), wherein the head part is rotatable around an axis of rotation that is substantially parallel to, but not coincident with the longitudinal centre axis of the probe.
s) A probe according to aspect r) and comprising any of the elements of aspects a)-o).
t) Use of a probe according to any of aspects a)-s) for the examination and surgical treatment of the anal canal of a mammalian body.
u) A method of treating anal incontinence comprising the use of the probe according to any of aspects a)-s).
v) A method according to aspect u) comprising;
   inserting the probe into the anal canal of a patient, and
   injecting an agent into the part of the anal canal, which has entered the groove(s) of the probe.
w) A method according to aspect v), further comprising, prior to the step of injecting and after the step of inserting;
   turning the probe until the part of the anal canal to be examined or treated is aligned with one or more of the grooves of the probe, and
   retracting the removable element(s) filling out the groove(s) so as to allow the part of anal canal to enter the groove(s).
x) A method according to any of aspects v)-w), further comprising, prior to the step of injecting;
   rotating the head part into a displaced position in relation to the other part of the probe,
   retracting the probe out of the anal canal until said head part abuts the tissue of the anal canal forming the junction to the rectum, and
   measuring the length of the anal canal by means of the measuring line on the probe.
y) A kit comprising (1) a probe according to any of aspects a)-s), (2) a material suitable for injection into the human body, and (3) surgical instruments for injecting said material.

1. A probe for insertion in a patients anal canal and comprising a hollowed tubular member having a closed end adapted to enter the anal canal and an oppositely arranged open end for receiving surgical instruments, at least one longitudinally extending groove being provided in the sidewall of the hollowed member so as to allow a part of the patients anal canal to enter into the groove, said groove being accessible for examination and treatment of said part of the anal canal via an opening near said open end of the hollowed member.

2. A probe according to claim 1 and comprising a rotatable head part defined by or at said closed end and being rotatable around an axis of rotation that is substantially parallel to, but not coincident with the longitudinal centre axis of the probe.

3. A probe according to claim 2 and comprising a handle near said open end for rotating said head part.

4. A probe according to claim 3, wherein the handle is connected to the rotatable head part via a shaft suspended inside the hollowed tubular member.

5. A probe according to claim 1 comprising a removable element positioned in said groove for filling out the groove during insertion and movement of the probe in the anal canal.

6. A probe according to claim 1 comprising a widened portion or a collar arranged near said open end for defining an insertion stop for the probe.

7. A probe according to claim 1 comprising two or three or four or more grooves distributed along the circumference of the hollowed member, each extending longitudinally in said sidewall so as to allow parts of the patients anal canal to enter into the groove for examining and treatment.

8. A probe according to claim, wherein the closed end or head part has a substantially cone-shaped form for easing insertion.

9. A probe according to claims 1, wherein the probe is substantially cylindrical with an outer diameter of between a 20 mm and 30 mm.

10. A probe according to claim 1, wherein the groove(s) extends substantially from the closed end or head part to the open end.

11. A probe according to claim 1, wherein the width of the groove(s) is between 1 mm and 5 mm, preferably 3 mm.

12. A probe according to any claim 1, wherein the surgical instruments comprise scopes, ultrasound probes and/or needles.

13. A probe according to claims 1, said probe being made of plastic.

14. A probe according to claim 13, wherein the probe or at least the groove(s) is made of transparent plastic.

15. A probe according to claim 1 comprising a measuring line on its outer surface for measuring the length of the anal canal.

16. A probe for insertion in a patients anal canal and comprising a hollowed tubular member with an open end for receiving surgical instruments, a head part arranged opposite to said open end and being adapted to enter the anal canal, at least one longitudinally extending groove being provided in the sidewall of the hollowed member so as to allow a part of the patients anal canal to enter into the groove, said groove being accessible for examination and treatment of said part of the anal canal via an opening near said open end of the hollowed member.

17. A probe according to claim 16, wherein said head part is defined by a closed end of said hollowed member.

18. A probe according to claim 16, wherein the head part is rotatable around an axis of rotation that is substantially parallel to, but not coincident with the longitudinal centre axis of the probe.

19. Use of a probe according to claim 1 for the examination and surgical treatment of the anal canal of a mammalian body.

20. Use of a probe according to claims 16 for the examination and surgical treatment of the anal canal of a mammalian body.

21. A method of treating anal incontinence comprising the use of the probe according to claim 1.

22. A method of treating anal incontinence comprising the use of the probe according to claim 16.

23. A method according to claim 21 comprising;
inserting the probe into the anal canal of a patient, and
injecting an agent into the part of the anal canal, which has entered the groove(s) of the probe.

24. A method according to claim 23, further comprising, prior to the step of injecting and after the step of inserting;
turning the probe until the part of the anal canal to be examined or treated is aligned with one or more of the grooves of the probe, and
retracting the removable element(s) filling out the groove(s) so as to allow the part of anal canal to enter the groove(s).

25. A method according claim 23, further comprising, prior to the step of injecting;
rotating the head part into a displaced position in relation to the other part of the probe,
retracting the probe out of the anal canal until said head part abuts the tissue of the anal canal forming the junction to the rectum, and
measuring the length of the anal canal by means of the measuring line on the probe.

26. A kit comprising i) a probe according to claim 1, ii) a material suitable for injection into the human body, and iii) surgical instruments for injecting said material.
